(19) United States
(12) Patent Application Publication
(10) Pub. No.: US 2006/0134802 A1
(43) Pub. Date: Jun. 22, 2006

(54) Solid-phase immunochromatographic methods

(75) Inventors: Raphael Donati, Radon (FR); Patrick Bigot, Ecouche (FR)

Correspondence Address: OLIFF & BERRIDGE, PLC, P.O. BOX 19928, ALEXANDRIA, VA, 22320, US

(73) Assignee: VEDALAB, Alencon (FR)

(21) Appl. No.: 10/550,317

(22) PCT Filed: Mar. 26, 2004
(86) PCT No.: PCT/FR04/00775

§ 371 Date: Sep. 22, 2005

(30) Foreign Application Priority Data
Mar. 28, 2003 (FR) 03/03877

(51) Int. Cl. G01N 33/558

(52) U.S. Cl. 436/514

(57) ABSTRACT

The invention concerns solid-phase immunochromatographic methods for detecting an analyte in a liquid sample. In the inventive methods, a binding reagent conjugated with a particulate marker is extemporaneously added in liquid form.

The present invention relates to a method for detecting an analyte in a liquid sample.

Solid-phase immunochromatographic assays are well known to those skilled in the art. These assays use a porous solid support within which the sample and the reagents migrate by capillary diffusion. Devices in which the solid support is in the form of a “dip-stick” are in particular known. These assays use a solid chromatographic support comprising a detection zone on which a capture reagent specific for the analyte is immobilized. This solid support is brought into contact with a solution comprising, firstly, the sample to be tested and, secondly, a labeled binding reagent specific for the analyte. This solution migrates, by capillary diffusion, in the solid support as far as the zone bearing the immobilized capture reagent. In a sandwich assay, the labeled binding reagent binds to the analyte, while the latter is immobilized on the solid support by the capture reagent. The presence or absence of the analyte in the sample is thus measured through the detection of the labeled reagent.

EP 0 284 232 describes solid-phase immunochromatographic assays in which the solid support directly bears, in lyophilized or dehydrated form, the binding reagent conjugated to a particulate label. The reagent conjugated with the particulate label is immobile in lyophilized form, but becomes mobile in the solid support in the moist state. Thus, when the solid support is brought into contact with a liquid sample, the latter migrates by capillary diffusion in the support, entraining the binding reagent conjugated to the particulate label. In these assays, it is not necessary to mix the conjugated reagent and the sample beforehand, and all the reagents necessary for the assay are therefore integrated into the solid support. In addition, the analyte-specific labeled binding reagent is labeled with a particulate label that can be detected by direct observation. No additional manipulation is therefore necessary in order to read the results of the assay. These assays therefore only require a small number of manipulations and are easy to use and rapid.

EP 0 291 194, EP 0 560 411 and EP 0 560 410 also describe test devices in which the binding reagent conjugated to a particulate label is carried by the solid support. In addition, in these devices, the solid support is incorporated into a casing provided with an opening for depositing the sample and with an observation window for reading the results. The casing facilitates gripping of the device and protects the solid support. In addition, those patents also describe devices in which one of the ends of the support protrudes from the casing so as to facilitate the depositing of the liquid sample. This protruding end of the solid support can thus be directly brought into contact with a stream of urine, for example.

WO 00/00288 describes improved devices comprising a casing and a solid support. The solid support is provided with a mobile collecting member for better collection of the sample.

EP-A1-0 458 231 relates to solid-phase immunoassays for detecting an analyte in a liquid sample. These assays use a solid support, typically a membrane, on which a reagent for capturing the analyte is immobilized. After the sample has been deposited, the analyte immobilized on the solid support is detected using a binding reagent conjugated to urease. That document describes methods in which the sample and then the labeled binding reagent are deposited on the solid support.

EP-A2-0 462 376 describes lateral flow immunochromatographic assays. The assays described use a binding reagent conjugated to a particulate label. That document describes assays in which the sample and the conjugated binding reagent are applied separately to the solid support or combined prior to the depositing on the support so as to form a test solution. Preferably, the conjugated binding reagent is incorporated into the chromatographic support.

U.S. Pat. No. 6,008,056 describes automated devices for lateral immunochromatography. The labeled binding reagent is incorporated into the solid support or mixed with the sample before or during deposition onto the chromatographic support.

EP-A1-1 020 726 describes lateral flow immunochromatographic assays. The sample is deposited onto the support before the labeled binding agent, or a mixture comprising the sample and one or more labeled reagents is deposited onto the support.

WO-A1-97 09620 describes methods for detecting an analyte in a sample by quantitative or semiquantitative immunochromatography. The labeled binding reagent is incorporated into the solid support.

However, these solid-phase immunochromatographic assays sometimes exhibit insufficient sensitivity and reproducibility. This problem arises more particularly for the detection of analytes present at a low concentration or with the detection of analytes in a sample of complex type, such as whole blood, for example. In addition, because of these deficiencies, such assays are only suitable for determining the absence or presence of an analyte in a sample, and only therefore enable a qualitative result to be obtained. Measurements that are more quantitative can only be made with difficulty. In addition, a considerable background noise and also a zone effect (or “Hook effect”) are commonly observed, which impair the readability of the result. The Hook effect is an undesirable effect well known in immunoassays. It occurs when the analyte is present in the sample at a very high concentration. The Hook effect can then produce a negative result, resulting in an aberrant conclusion that the analyte is absent from the sample.

In order to remedy these drawbacks, the present invention proposes solid-phase immunochromatographic methods that make it possible to obtain increased sensitivity and reproducibility while at the same time limiting the background noise and the Hook effects. The methods according to the invention are particularly suitable for samples of complex type, such as blood, for example. Given that the background noise is decreased while the sensitivity and reproducibility are increased, the methods of the present invention advantageously make it possible to detect several different analytes simultaneously on the same support. In addition, the analyte present in the liquid sample can be measured and quantified due to the performance levels of the methods according to the invention.

In the methods according to the present invention, the binding reagent conjugated to a particulate label is added extemporaneously in liquid form. Thus, in the methods according to the invention, the order of deposition of the sample and of the various reagents is of great importance for the effectiveness of the detection assay.

In a first embodiment, the method for detecting an analyte in a liquid sample according to the invention comprises the following steps:
a) a porous solid support provided with a collection zone and a detection zone is provided, a capture reagent being immobilized in the detection zone;
b) the following are deposited, separately and successively, in the collection zone of the porous solid support:
   i) a binding reagent conjugated to a particulate label, the reagent being in liquid form,
   ii) the liquid sample,
c) an amount of time sufficient for the migration, by capillary diffusion, of the binding reagent conjugated to a particulate label and of the liquid sample from the collection zone to the detection zone of the porous solid support is allowed to elapse,
d) the extent to which the reagent conjugated to a particulate label attaches in the detection zone is observed.

Advantageously, in step b), the liquid sample is deposited upstream of the binding reagent conjugated to a particulate label, relative to the direction of migration from the collection zone to the detection zone of the porous solid support.

The present invention also relates to a method for detecting an analyte in a liquid sample, comprising the following steps:
a) a porous solid support provided with a collection zone and a detection zone is provided, a capture reagent being immobilized in the detection zone;
b) the following are deposited, separately and successively, in the collection zone of the porous solid support:
   i) a binding reagent conjugated to a particulate label, the reagent being in liquid form,
   ii) the liquid sample,
   iii) a diluent in liquid form,
c) an amount of time sufficient for the migration, by capillary diffusion, of the reagent conjugated to a particulate label, of the liquid sample and of the diluent from the collection zone to the detection zone of the porous solid support is allowed to elapse,
d) the extent to which the binding reagent conjugated to a particulate label attaches in the detection zone is observed.

A subject of the present invention is also a method for detecting an analyte in a liquid sample, comprising the following steps:
a) a porous solid support provided with a collection zone and a detection zone is provided, a capture reagent being immobilized in the detection zone;
b) the following are deposited, separately and successively, in the collection zone of the porous solid support:
   i) the liquid sample,
   ii) a binding reagent conjugated to a particulate label, the reagent being in liquid form,
   iii) a diluent in liquid form,
c) an amount of time sufficient for the migration, by capillary diffusion, of the reagent conjugated to a particulate label, of the liquid sample and of the diluent from the collection zone to the detection zone of the porous solid support is allowed to elapse,
d) the extent to which the binding reagent conjugated to a particulate label attaches in the detection zone is observed.

Advantageously, in step b), the diluent in liquid form is deposited upstream of the binding reagent conjugated to a particulate label and upstream of the liquid sample, relative to the direction of migration from the collection zone to the detection zone of the porous solid support.

In a preferred embodiment of the invention, the binding reagent conjugated to a particulate label and the capture reagent immobilized in the detection zone make it possible to detect the analyte by means of a sandwich assay.

In another preferred embodiment of the invention, the binding reagent conjugated to a particulate label and the capture reagent immobilized in the detection zone make it possible to detect the analyte by means of a competition assay.

Preferably, the porous solid support is a porous solid support in the form of a chromatographic strip or narrow strip.

Advantageously, the porous solid support is integrated into a support to be gripped provided with at least one observation window for observing the extent to which the reagent conjugated to a particulate label attaches in the detection zone of the porous solid support.

In a particular embodiment of the invention, the support to be gripped is provided with at least one opening for depositing, respectively, the liquid sample, the binding reagent conjugated to a label and, where appropriate, the diluent, onto the collection zone of the porous solid support.

In an advantageous embodiment of the invention, the porous solid support is integrated into a support to be gripped provided with at least one observation window for observing the extent to which the reagent conjugated to a particulate label attaches in the detection zone of the porous solid support; the porous solid support being also provided with a first opening for depositing the binding reagent conjugated to a particulate label in the collection zone of the porous solid support and with a second opening, upstream of the first opening, for depositing the liquid sample in the collection zone of the porous solid support.

In another advantageous embodiment of the invention, the porous solid support is integrated into a support to be gripped provided with at least one observation window for observing the extent to which the reagent conjugated to a particulate label attaches in the detection zone of the porous solid support; the support to be gripped being also provided with a first opening for depositing the binding reagent conjugated to a particulate label and the sample in the collection zone of the porous solid support, and with a second opening, upstream of the first opening, for depositing the diluent in liquid form in the collection zone of the porous solid support.

Preferably, the support to be gripped consists of a casing.

A subject of the present invention is also a kit for detecting an analyte in a liquid sample, comprising a) a porous solid support provided with a collection zone and a detection zone, a capture reagent being immobilized in the detection zone, and b) a binding reagent conjugated to a particulate label in liquid form.

Advantageously, the kit for detecting an analyte in a liquid sample according to the invention also comprises a diluent.

Preferably, the porous solid support is integrated into a support to be gripped.

Preferably, the porous solid support is integrated into a support to be gripped provided with at least one observation window for observing the detection zone of the porous solid support.

In a preferred embodiment, the porous solid support is integrated into the support to be gripped provided with at least one opening for depositing the liquid sample and/or the binding reagent conjugated to a particulate label and/or the diluent in the collection zone of the porous solid support.

Preferably, the porous solid support is integrated into a support to be gripped in the form of a casing.

The term “analyte” is intended to mean any chemical, biochemical or biological entity that it is desired to detect in a sample. Among the analytes detected by means of the methods according to the present invention, mention will in particular be made of proteins, peptides, antibodies, hormones, steroids, antigens derived from infectious agents or from tumor cells, infectious agents such as bacteria, viruses or parasites, nucleic acids (DNA or RNA), therapeutic molecules, drugs or antibiotics.

The term “detecting” is intended to mean determining the presence or the absence of an analyte in a sample, but also measuring and quantifying an analyte in a sample. This is because the performance levels of the methods according to the invention make it possible to carry out quantitative or semiquantitative measurements.

In a particular embodiment of the invention, the analyte is hCG (chorionic gonadotropin hormone) or PSA (prostate specific antigen).

In another particular embodiment of the invention, the analyte is a nucleic acid. In this case, the nucleic acid present in the sample is preferably amplified beforehand according to techniques well known to those skilled in the art (PCR, etc.). Preferably, this amplification step also makes it possible to label the amplified nucleic acid using biotinylated primers or by incorporating biotin-labeled nucleotides, for example. Alternatively, the nucleic acid may, for example, be labeled with biotin at its 3′ end using an appropriate terminal transferase. Before being deposited, the sample is denatured, either by heat shock or in the presence of a solution 0.2 N NaOH, 0.05 M EDTA or any other appropriate method. This denaturation step makes it possible to obtain single-stranded nucleic acids.

The term “liquid sample” is intended to mean any sample in which the analyte being sought is in solution or in suspension. This liquid sample may in particular be any biological or body fluid. The liquid sample may also have been obtained directly or indirectly from a biological or body fluid. The sample may also be a liquid extract of a solid sample.

In a preferred embodiment of the invention, the liquid sample is urine, whole blood, plasma or serum.

The reagents used in the methods according to the present invention are well known to those skilled in the art.

The binding reagent conjugated to a particulate label and the capture reagent are specific for the analyte being sought in the sample.

In a particular embodiment of the invention, the binding reagent conjugated to a particulate label and the capture reagent immobilized in the detection zone of the solid support make it possible to detect the analyte by means of a sandwich assay.

In another particular embodiment of the invention, the binding reagent conjugated to a particulate label and the analyte-specific capture reagent immobilized in the detection zone of the solid support make it possible to detect the analyte by means of a competition assay.

Sandwich assays and competition assays are well known to those skilled in the art. In a sandwich assay, the analyte-specific capture reagent and the labeled binding reagent are predetermined so as to bind respectively and specifically with the analyte, for example on two identical or different epitope sites of the analyte. In a competition assay, the labeled binding reagent is identical or similar to the analyte, so as to bind with the capture reagent, in competition with the analyte.

The term “capture reagent” is intended to mean any chemical, biochemical or biological entity capable of binding specifically with the analyte.

In the case of a competition assay, the capture reagent also binds to the binding reagent. The analyte and the capture reagent typically form a ligand/anti-ligand, antigen/antibody, DNA/RNA or DNA/DNA couple. Thus, if the analyte is an antigen or a hapten, the capture reagent is, for example, an antibody specific for the analyte. If the analyte is an antibody, the capture reagent is the antigen recognized by the antibody or an antibody that specifically recognizes the analyte. If the analyte is a nucleic acid, the capture reagent is, for example, a complementary DNA probe.

The immobilized capture reagent is preferably a polyclonal or monoclonal antibody having a high affinity for the analyte, and it is more preferably a monoclonal antibody.

The analyte-specific capture reagent is immobilized on the solid support according to techniques known to those skilled in the art. This capture reagent is immobilized in such a way that it is not mobile in the moist state. This immobilization may be carried out, for example, by absorption or by covalent coupling. When the capture reagent is a nucleic acid, it is, for example, attached to a support of nitrocellulose type by UV treatment or by any other technique known to those skilled in the art.

The term “binding reagent” is intended to mean any chemical, biochemical or biological entity capable of binding specifically with the analyte or with the capture reagent in competition with the analyte.

The term “bind” or “binding” is intended to mean any strong binding, for example covalent, or any weak binding, for example of the antigen/antibody or avidin/streptavidin type.

The binding reagent is, for example, an antibody, an antigen or a nucleic acid.

Any other binding reagent known to those skilled in the art may be used, such as one (or more) anti-biotin monoclonal antibody (or antibodies), avidin, streptavidin or polystreptavidin. These reagents may be natural or recombinant.

In a competition assay, the binding reagent is, for example, the analyte itself or an appropriate analog of the analyte. The expression “appropriate analog of the analyte” is intended to mean an analog that binds specifically to the analyte-specific capture reagent. The labeled binding reagent is therefore the analyte conjugated to a particulate label or an analog of the analyte conjugated to a particulate label.

In a sandwich assay, the binding reagent binds specifically to the analyte. The labeled binding reagent is therefore, for example, an antibody specific for the analyte, conjugated to a particulate label.

When the analyte is a biotin-labeled nucleic acid, the binding reagent is typically an anti-biotin antibody conjugated to a particulate label such as colloidal gold, for example.

The binding reagent is conjugated to a particulate label that allows direct measurement or observation of the result of the test. The particulate label can be observed directly with the naked eye when it is concentrated in the detection zone of the solid support. The particulate label may be measured directly with the naked eye or using a measuring device. This measurement is carried out by direct observation that does not require any additional handling. Typically, the particulate labels consist of small particles that are water-insoluble and that therefore form liquid-phase suspensions, i.e. a dispersion of solid particles in a liquid.

The particulate labels are well known to those skilled in the art. Colored or fluorescent particulate labels are in particular known. By way of example, mention will be made of colloidal gold, colored latex particles, fluorescent latex particles and particles conjugated to avidin or to streptavidin.

Among the labels for direct observation with the naked eye, mention will also be made of dextran-type labels (Hansen T. M., IVD Technology 4, 35-40, 2003). The binding reagent is then conjugated to a dextran chain (polysaccharide derivative) bearing fluorophores.

The binding reagent is conjugated to the particulate label according to known techniques.

In the methods according to the present invention, the binding reagent conjugated to a particulate label is in liquid form.

The term “reagent in liquid form” is intended to mean any reagent in which the binding reagent is in solution or in suspension. The preparation of the binding reagent conjugated to the particulate label in liquid form is carried out according to techniques described in the literature. Usually, the conjugated binding reagent is in solution or in suspension in a buffered saline solution. This solution may also comprise stabilizers and other compounds, such as antibacterial or antifungal agents. Among stabilizers, mention will, for example, be made of bovine serum albumin (BSA) and casein.

In certain methods according to the present invention, a diluent is used when the liquid sample is plasma, serum or whole blood, for example. This diluent migrates in the solid support, entraining the sample and the labeled binding reagent. Typically, this diluent is composed of a buffered saline solution; it may also comprise a detergent or any other component required for the reaction.

In the methods for detecting nucleic acids, the diluent may consist of a hybridization buffer. Such hybridization buffers are well known to those skilled in the art.

The porous solid supports used in the immunochromatographic assays according to the invention are well known to those skilled in the art (EP 0 284 232). The porosity of the support allows capillary diffusion of the sample and of the reagents in the liquid or moist state.

By way of example, the porous solid support may consist of various chromatographic supports, of cellulose, of nylon, of nitrocellulose, of polyethylene or of glass fiber.

The solid support may consist of one or more different parts. The various parts of the support may consist of different materials. When the solid support consists of various parts or of various materials, these elements are arranged so as to allow continuity of the capillary flow in the solid support.

Preferably, the porous solid support is a porous solid support in the form of a chromatographic strip or narrow strip.

The solid support may thus be in the form of a chromatographic strip consisting of several superimposed or overlapping narrow strips.

Typically, the porous solid support comprises a zone for collecting the sample and a detection zone carrying the capture reagent. These zones are arranged so as to allow continuity of the capillary flow from the collection zone to the detection zone. The collection zone and the detection zone are two distinct and separate zones of the porous solid support. The sample, the labeled binding reagent and, where appropriate, the diluent are deposited in the collection zone and migrate through the porous solid support to the detection zone. The porous solid support thus consists, for example, of a chromatographic strip, one of the ends of which constitutes the collection zone, the detection zone being located in proximity to the other end of the strip.

These zones may, for example, be present in the same plane on a strip consisting of a single material. Advantageously, a specific material corresponds to each zone of the solid support. A porous absorbent material may, for example, be used for the sample collection zone.

The sample collection zone of the solid support may thus consist of a collecting member made of absorbent material. This collecting member may be directly brought into contact with a stream of urine, for example. The solid support may also comprise a mobile collecting member as described in WO 00/00288.

The detection zone of the porous solid support may also comprise a second capture reagent immobilized on the porous support downstream of the first capture reagent. This second immobilized capture reagent makes it possible to check that the test is progressing correctly by verifying, for example, the migration of the binding reagent conjugated to the particulate label in the solid support. The second capture reagent is, for example, an antibody specific for the binding reagent.

In the methods according to the present invention, the labeled binding reagent, the sample and, where appropriate, the diluent are deposited separately, successively and in liquid form in the collection zone of the solid support. Extemporaneous deposition of the labeled binding reagent in liquid form before the sample and/or before the diluent makes it possible to decrease background noise and Hook effect while at the same time increasing sensitivity, due to the immediate and complete contact between the sample and the labeled binding reagent. The reproducibility of the methods according to the invention is also considerably increased by the fact that the dose of labeled binding reagent added in liquid form is precise.

In addition, in the methods according to the present invention, the extemporaneous deposition of the labeled binding reagent in liquid form before the sample and/or before the diluent makes it possible to obtain a washing effect that results in a decrease in the background noise and in the Hook effect. This is particularly advantageous for the detection of analytes in a complex sample such as blood, for example.

Advantageously, the sample or the diluent is deposited in the collection zone upstream of the labeled binding reagent.

In order to control the amount of labeled binding reagent deposited on the collection zone of the porous solid support, this deposition is preferably carried out using a pipette, a dropper or a dropper bottle.

The liquid sample can also be deposited using a pipette, a dropper or a dropper bottle. In another embodiment, the deposition of the sample is carried out by soaking the collection zone of the solid support in the liquid sample. When the liquid sample is urine, the collection zone of the solid support can also be directly brought into contact with a stream of urine.

In a preferred embodiment of the invention, the porous solid support is integrated into a support to be gripped. This support to be gripped can partially or completely envelop the porous solid support. Usually, the support to be gripped is in the form of a casing.

These supports to be gripped or casings are in particular described in EP 0 291 194, EP 0 560 411, EP 0 560 410 and in WO 00/00288.

The support to be gripped facilitates the handling of the porous solid support and protects it against moisture in particular.

The support to be gripped can consist of various materials such as cardboard, plasticized cardboard or, more preferably, plastics. Advantageously, the support to be gripped consists of a rigid and impermeable material.

Typically, the support to be gripped is provided with at least one observation window for observing the detection zone of the porous solid support.

In one embodiment of the invention, the porous solid support can comprise a collection zone that protrudes from the support to be gripped, for depositing the liquid sample and/or the labeled binding reagent and/or the diluent.

In another embodiment of the invention, the support to be gripped or the casing comprises at least one opening for depositing the liquid sample and/or the labeled binding reagent and/or the diluent in the collection zone of the porous solid support.

The invention will be understood more clearly from the figures and examples below:

Figures

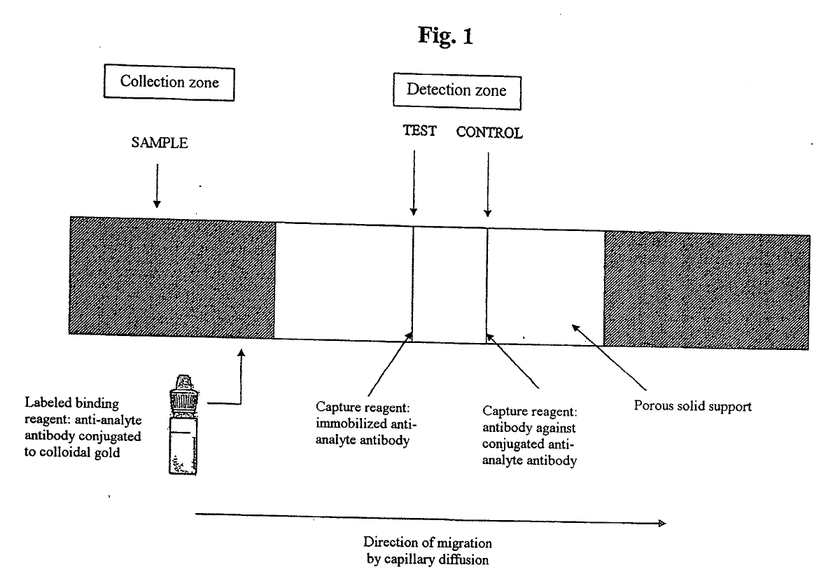

FIG. 1: Principles of the immunochromatographic methods of the invention

The shaded regions represent parts of the solid support that consist of an absorbent material.

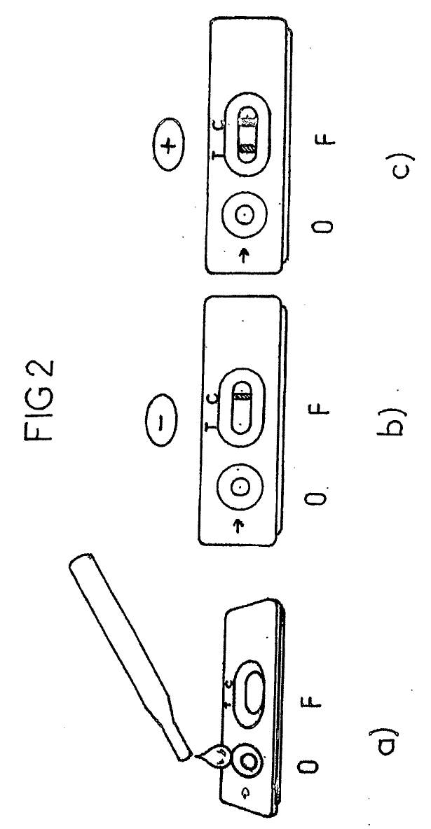

FIG. 2: Immunochromatographic assay device

FIG. 2 represents a device comprising a casing comprising a porous solid support. The casing is provided with an opening (O) for depositing the liquids and with an observation window (F). FIG. 2a represents the deposition of the liquids onto the solid support via the opening in the casing. FIG. 2b shows a negative result that is visible through the observation window (F). FIG. 2b shows a positive result for a sandwich assay that is visible through the observation window (F).

T=test line, C=control line, O=opening, F=observation window.

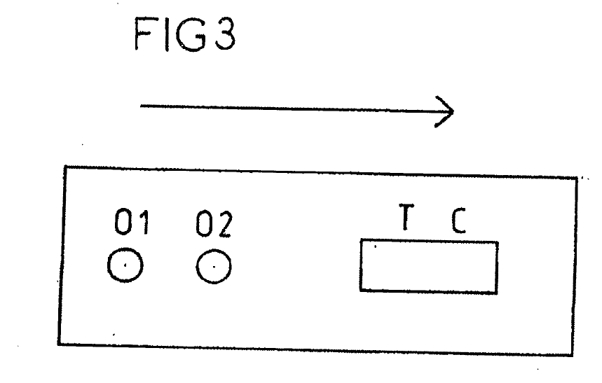

FIG. 3: Two-well immunochromatographic assay device FIG. 3 represents a casing comprising a porous solid support. The casing is provided with two distinct openings (O1 and O2) for depositing the liquids and with an observation window. The arrow indicates the direction of migration by capillary diffusion.

O1=opening No. 1, O2=opening No. 2, T=test line, C=control line.

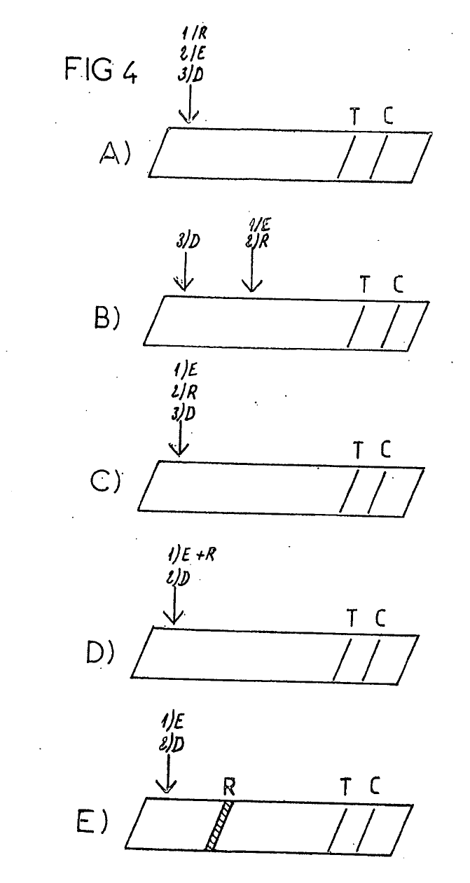

FIG. 4: Comparative examples with diluent

R=labeled binding reagent, E=sample, D=diluent, T=test line, C=control line.

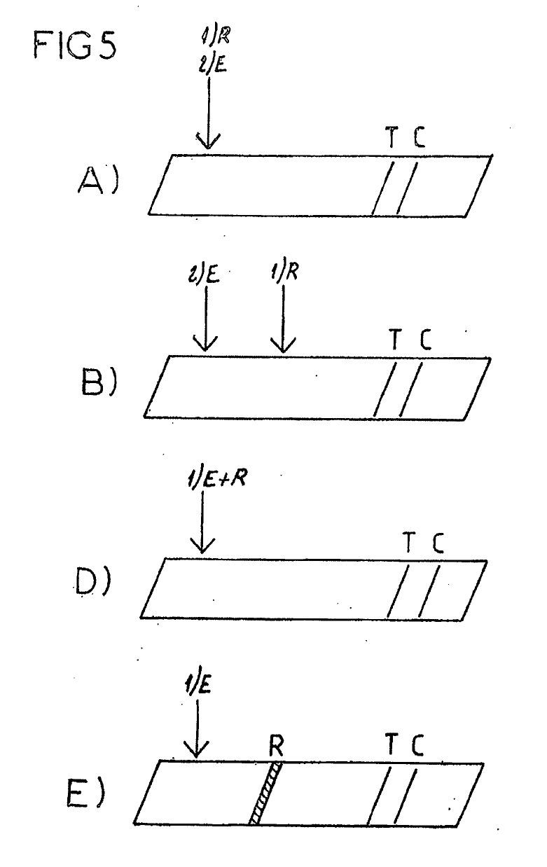

FIG. 5: Comparative examples without diluent

R=labeled binding reagent, E=sample, T=test line, C=control line

### EXAMPLES

### Example 1

### Immunochromatographic Methods with Diluent Device

The methods were carried out with the devices represented in FIG. 2 and FIG. 3.

Analyte and Sample

The analyte is prostate specific antigen (PSA) detected in serum. The test could be carried out in the same way with whole blood or plasma.

Reagents and Diluent

The labeled binding reagent is an anti-PSA monoclonal or polyclonal antibody conjugated with colloidal gold in a buffer (0.1M PBS, pH 8) containing bovine serum albumin (1% BSA) as stabilizer.

A first capture reagent is immobilized at the test line of the detection zone. This first capture reagent is an anti-PSA monoclonal or polyclonal antibody.

A second capture reagent is immobilized at the control line of the detection zone. This second capture reagent is a monoclonal or polyclonal antibody directed against the antibody of the labeled binding reagent.

The diluent consists of a PBS buffer (0.1M, pH 8) containing a detergent (0.05% Tween 20).

Methods

The various methods that were compared are represented in FIG. 4. Methods A, B and C are in accordance with the invention. In all cases, the amount of sample and the amount of binding reagent conjugated to the particulate label, per test, were identical whatever the method considered, so as not to distort the results.

Sample Volume=25 μl (Version A, B, C, D, E)

Diluent volume=100 μl (Version A, B, C, D), 150 μl (version E).

Labeled binding reagent volume=35 μl (version A, B, C, D), identical but in dehydrated form (version E).

The methods were carried out in the following way:

Version A

1) labeled binding reagent

2) sample

3) diluent

Version B

1) sample in opening 2

2) labeled binding reagent in opening 2

3) diluent in opening 1 (upstream of opening 2).

Version C

1) sample

2) labeled binding reagent

3) diluent.

Version D

1) sample and labeled binding reagent premixed

2) diluent.

Version E

1) sample

2) diluent.

In the latter method, the labeled binding reagent is directly carried by the solid support.

Results

The performance levels obtained for each of the methods were measured and quantified using a reflectometer. The Hook effect is evaluated using a very concentrated sample of analyte.

| | Background | Hook | | |
|---|---|---|---|---|
| Method | noise | effect | Sensitivity | Reproducibility |
| A | 4 | 5 | 5 | 4 |
| B | 5 | 3 | 4 | 4 |
| C | 4 | 4 | 3 | 4 |
| D | 4 | 4 | 2 | 4 |
| E | 3 | 2 | 1 | 2 |

Classification from 1 to 5 (1 is the least effective, 5 is the most effective).

### Example 2

### Immunochromatographic Methods Without Diluent

Device

The methods were carried out with the devices represented in FIG. 2 and in FIG. 3.

Analyte and Sample

The analyte is chorionic gonadotropin hormone (hCG) detected in the urine. The test could be carried out in the same way with serum or plasma.

Reagents

The labeled binding reagent is an anti-hCG monoclonal or polyclonal antibody conjugated with colloidal gold in a buffer (0.1M PBS, pH 8) containing bovine serum albumin (1% BSA) as stabilizer.

A first capture reagent is immobilized at the test line of the detection zone. This first capture reagent is an anti-hCG monoclonal or polyclonal antibody.

A second capture reagent is immobilized at the control line of the detection zone. This second capture reagent is a monoclonal or polyclonal antibody directed against the labeled binding reagent antibody.

Methods

The various methods that were compared are represented in FIG. 5.

Methods A and B are in accordance with the invention. In all cases, the amount of sample and the amount of binding reagent conjugated to the particulate label, per test, were identical whatever the method considered, so as not to distort the results.

Sample volume=100 μl (version A, B, D); 100 μl+35 μl (version E)

labeled binding reagent volume=35 μl (version A, B, D), identical but in dehydrated form (version E).

The methods were carried out in the following way:

Version A

1) labeled binding reagent

2) sample

Version B

1) labeled binding reagent in opening 2

2) sample in opening 1

Version D

1) sample and labeled binding reagent premixed.

Version E

1) sample

In the latter method, the labeled binding reagent is directly carried by the solid support.

Results

The performance levels obtained for each of the methods were measured and quantified using a reflectometer. The Hook effect is evaluated using a very concentrated sample of analyte.

| | Background | Hook | | |
|---|---|---|---|---|
| Method | noise | effect | Sensitivity | Reproducibility |
| A | 4 | 4 | 4 | 4 |
| B | 5 | 5 | 1 | 4 |
| D | 3 | 1 | 5 | 4 |
| E | 3 | 3 | 3 | 2 |

Classification from 1 to 5 (1 is the least effective, 5 is the most effective).

### Example 3

### Method for Detecting Morphine (Competition Assay)

Device

This method was carried out with the device represented in FIG. 2.

Analyte and Sample

The analyte is morphine detected in the urine.

Reagents

The labeled binding reagent is a morphine hapten-BSA conjugated to particles of colloidal gold in a buffer (0.1M PBS, pH 8) containing bovine serum albumin (1% BSA) as stabilizer.

A capture reagent is immobilized at the test line of the detection zone. This first capture reagent is an anti-morphine monoclonal antibody.

Method

35 μl of binding reagent conjugated to the particulate label are deposited and then 150 μl of urine are deposited in the same well of the casing. 5 minutes later, the results are read.

Results

Compared with a test using a solid support carrying the conjugated binding reagent in dehydrated form, the background noise is decreased and the reproducibility is improved.

### Example 4

### Method for Detecting a Nucleic Acid

Device

This method is carried out with the device represented in FIG. 2.

The device comprises a nitrocellulose narrow strip to which a probe specific for a nucleic acid of E. coli or of Chlamydia is attached (by UV treatment or by any other technique). The narrow strip is embedded in a plastic casing provided with an opening for depositing the reagents and with an observation window.

Analyte and Sample

The analyte being sought in the sample is a DNA or an RNA of E. coli or of Chlamydia. The nucleic acid is amplified beforehand according to conventional methods such as, for example, PCR. The DNA is labeled by means of biotinylated primers or by incorporation of biotin-labeled nucleotides. Alternatively, the 3′ end of the nucleic acids is labeled with biotin using a terminal transferase.

Reagents

The labeled binding reagent is an anti-biotin rabbit polyclonal antibody labeled with colloidal gold.

The hybridization buffer is a PBS buffer containing 0.1% of Tween 20. Other hybridization buffers can be used.

Method

The amplified and biotin-labeled nucleic acids (sample) are denatured either by heat shock, or in the presence of 1.2N NaOH, 0.05M EDTA.

25 μl of denatured sample are deposited in the sample well (opening).

40 μl of labeled binding reagent (anti-biotin conjugate) are then deposited in the sample well. These first two steps can be inverted.

150 μl to 200 μl of hybridization buffer are then deposited in the sample well.

Results

When the nucleic acid being sought is present in the sample, it migrates by diffusion from the collection zone to the detection zone, where it attaches to the probe immobilized on the support. A red band appears in the test zone (test is read 10 to 20 minutes after the deposits). The control line also appears in the detection zone and shows that the reagents are functioning correctly.

1-13. (canceled)

14. A method for detecting an analyte in a liquid sample, comprising the following steps:
a) a porous solid support provided with a collection zone and a detection zone is provided, a capture reagent being immobilized in the detection zone;
b) the following are deposited, separately, successively and extemporaneously, in the collection zone of the porous solid support:
i) a binding reagent conjugated to a particulate label, the reagent being in liquid form,
ii) the liquid sample,
c) an amount of time sufficient for the migration, by capillary diffusion, of the binding reagent conjugated to a particulate label and of the liquid sample from the collection zone to the detection zone of the porous solid support is allowed to elapse,
d) the extent to which the binding reagent conjugated to a particulate label attaches in the detection zone is observed.

15. The method as claimed in claim 14, in which, in step b), the liquid sample is deposited upstream of the binding reagent conjugated to a particulate label, relative to the direction of migration from the collection zone to the detection zone of the porous solid support.

16. A method for detecting an analyte in a liquid sample, comprising the following steps:
a) a porous solid support provided with a collection zone and a detection zone is provided, a capture reagent being immobilized in the detection zone;
b) the following are deposited, separately, successively and extemporaneously, in the collection zone of the porous solid support:
i) a binding reagent conjugated to a particulate label, the reagent being in liquid form,
ii) the liquid sample,
iii) a diluent in liquid form,
c) an amount of time sufficient for the migration, by capillary diffusion, of the binding reagent conjugated to a particulate label, of the liquid sample and of the diluent from the collection zone to the detection zone of the porous solid support is allowed to elapse,
d) the extent to which the binding reagent conjugated to a particulate label attaches in the detection zone is observed.

17. A method for detecting an analyte in a liquid sample, comprising the following steps:
a) a porous solid support provided with a collection zone and a detection zone is provided, a capture reagent being immobilized in the detection zone;
b) the following are deposited, separately, successively and extemporaneously, in the collection zone of the porous solid support:
i) the liquid sample,
ii) a binding reagent conjugated to a particulate label, the reagent being in liquid form,
iii) a diluent in liquid form,
c) an amount of time sufficient for the migration, by capillary diffusion, of the binding reagent conjugated to a particulate label, of the liquid sample and of the diluent from the collection zone to the detection zone of the porous solid support is allowed to elapse,
d) the extent to which the binding reagent conjugated to a particulate label attaches in the detection zone is observed.

18. The method as claimed in claim 16, in which, in step b), the diluent in liquid form is deposited upstream of the binding reagent conjugated to a particulate label and upstream of the liquid sample, relative to the direction of migration from the collection zone to the detection zone of the porous solid support.

19. The method as claimed in claim 14, in which the binding reagent conjugated to a particulate label and the capture reagent immobilized in the detection zone make it possible to detect the analyte by means of a sandwich assay.

20. The method as claimed in claim 14, in which the binding reagent conjugated to a particulate label and the capture reagent immobilized in the detection zone make it possible to detect the analyte by means of a competition assay.

21. The method as claimed in claim 14, in which the porous solid support is a porous solid support in the form of a chromatographic strip or narrow strip.

22. The method as claimed in claim 14, in which the porous solid support is integrated into a support to be gripped provided with at least one observation window for observing the extent to which the reagent conjugated to a particulate label attaches in the detection zone of the porous solid support.

23. The method as claimed in claim 22, in which the support to be gripped is provided with at least one opening for depositing, respectively, the liquid sample, the binding reagent conjugated to a label and, where appropriate, the diluent, in the collection zone of the porous solid support.

24. The method as claimed in claim 15, in which the porous solid support is integrated into a support to be gripped provided with at least one observation window for observing the extent to which the reagent conjugated to a particulate label attaches in the detection zone of the porous solid support; the porous solid support being also provided with a first opening for depositing the binding reagent conjugated to a particulate label in the collection zone of the porous solid support and with a second opening, upstream of the first opening, for depositing the liquid sample in the collection zone of the porous solid support.

25. The method as claimed in claim 18, in which the porous solid support is integrated into a support to be gripped provided with at least one observation window for observing the extent to which the reagent conjugated to a particulate label attaches in the detection zone of the porous solid support; the support to be gripped being also provided with a first opening for depositing the binding reagent conjugated to a particulate label and the sample in the collection zone of the porous solid support, and with a second opening, upstream of the first opening, for depositing the diluent in liquid form in the collection zone of the porous solid support.

26. The method as claimed in claim 22, in which the support to be gripped consists of a casing.
